(19) United States
(12) Patent Application Publication
(10) Pub. No.: US 2014/0073042 A1
(43) Pub. Date: Mar. 13, 2014

(54) FLUIDIC DEVICE, CHEMICAL REACTION SYSTEM, AND NUCLEIC-ACID ANALYZING SYSTEM

(71) Applicant: Eishi Igata, Utsunomiya-shi (JP)

(72) Inventor: Eishi Igata, Utsunomiya-shi (JP)

(73) Assignee: CANON KABUSHIKI KAISHA, Tokyo (JP)

(21) Appl. No.: 14/115,981

(22) PCT Filed: Apr. 23, 2012
(86) PCT No.: PCT/JP2012/061504

§ 371 Date: Nov. 6, 2013

(30) Foreign Application Priority Data
May 10, 2011 (JP) 2011-105634

(51) Int. Cl. C12Q 1/68

(52) U.S. Cl. CPC C12Q 1/686
USPC 435/287.2; 435/289.1

(57) ABSTRACT

A fluidic device in which a first member and a second member are connected by an expanding member is provided. The fluidic device includes a first member having a fluid channel communicating with an opening; and a second member having a supply channel supplying a fluid to the fluid channel through the opening. An expanding member having a through-hole is fit into the opening, the fluid channel and the supply channel communicate with each other through the through-hole in the expanding member, and the first member and the second member are connected to each other with at least the expanding member being in an expanded state.

### TECHNICAL FIELD

The present invention relates to a fluidic device.

### BACKGROUND ART

Acquiring desired information, such as concentration or chemical composition, for checking the processes and results of chemical and biochemical reactions is the basics of analytical chemistry. Various apparatuses and sensors have been invented to acquire such information.

The size of such apparatuses and sensors has been reduced through high-precision processing and/or using semiconductor producing apparatuses, and research has been conducted to realize the entire information acquisition process on a device. That is, a concept known as a micro total analysis system (μ-TAS) or a lab-on-a-chip has been established. The goal for this is to carry out a process of analyte refinement or a chemical reaction by passing unrefined analytes or raw materials through a fluid channel or a microspace formed in a device and to acquire data on the concentration and/or chemical composition of the end product.

Since minutely small doses of solutions and gases are processed in devices used for such analysis and reaction, those devices are referred to as microfluidic devices.

Since the volume of the fluid retained in a microfluidic device is smaller than other conventional desktop-size analytical instruments, a reduction in the amount of reagent and a reduction in the amount of reaction time are expected as a result of a reduction in the amount of analytes. Technological development associated with μ-TAS has been achieved as the advantages of such fluidic devices have become recognized.

Although the advantages of analytical instruments using micro fluidic devices are recognized, such advantages can be achieved only when analytes and reagents for the analysis are supplied to microspaces in the devices. To supply a desired solution to a microspace in a fluidic device, the solution is injected from an opening in the device surface through a tube or capillary.

PTL 1 discloses a technique for joining devices by providing a hollow male joining part at an opening in a microfluidic device and a hollow female joining part at an opening in another micro fluidic device and connecting these joining parts.

### CITATION LIST

### Patent Literature

PTL 1 Japanese Patent Laid-Open No. 2001-194373

In the devices described in PTL 1, when a force is applied in one direction to the connected joining parts, the joining parts may be separated, or when the size of the opening is different from the size of the opening to be connected, the connection may become unstable. Fluids may leak from devices having an unstable connection.

Thus, the present invention provides a fluidic device that suppresses leakage from joining parts.

### SUMMARY OF INVENTION

The present invention provides a fluidic device including a first member having a fluid channel communicating with an opening; and a second member having a supply channel supplying a fluid to the fluid channel through the opening, wherein an expanding member having a through-hole is fit into the opening, the fluid channel and the supply channel communicate with each other through the through-hole in the expanding member, and the first member and the second member are connected to each other with at least the expanding member being in an expanded state.

According to the present invention, even when there is a gap between the first member and the second member, the fluid channel and the supply channel are stably connected, suppressing external fluid leakage.

### BRIEF DESCRIPTION OF DRAWINGS

FIG. 1 is a conceptual diagram of an embodiment of the present invention.

FIGS. 2A and 2B are conceptual diagrams illustrating the principle of the present invention.

FIGS. 3A and 3B are conceptual diagrams of an embodiment of the present invention in which fluidic chips are connected with an expanding member according to the present invention.

FIG. 4 is a conceptual diagram of an embodiment of the present invention in which two or more openings in a fluidic chip are connected with an expanding member according to the present invention.

FIG. 5 is a conceptual diagram of a fluidic device according to an embodiment of the present invention to be used in a high-temperature environment, the temperature being higher than room temperature.

FIGS. 6A and 6B are conceptual diagrams illustrating an expanding member and an opening shape according to an embodiment of the present invention.

### DESCRIPTION OF EMBODIMENTS

The present invention provides a fluidic device including a first member having a fluid channel communicating with an opening, and a second member having a supply channel supplying a fluid to the fluid channel through the opening, wherein an expanding member having a through-hole is fit into the opening, the fluid channel and the supply channel communicate with each other through the through-hole in the expanding member, and the first member and the second member are connected to each other with at least the expanding member being in an expanded state.

### First Embodiment

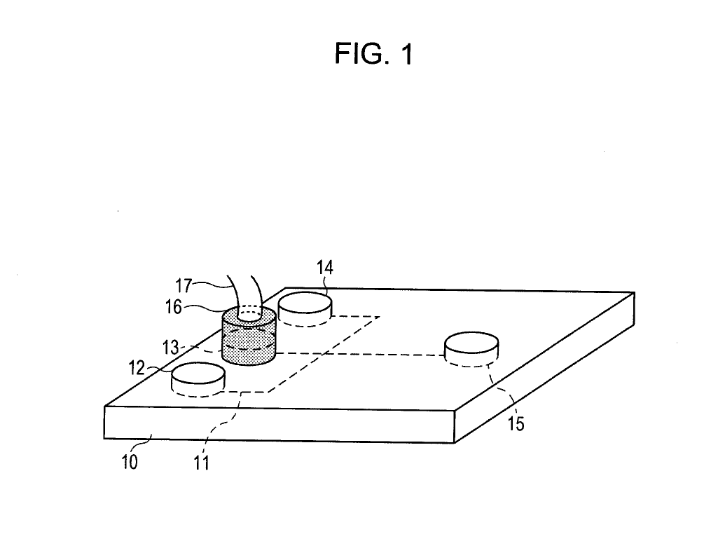

A fluidic device according to a first embodiment will be described with reference to FIG. 1.

The fluidic device according to the first embodiment includes at least a first member, a second member, and an expanding member.

As illustrated in FIG. 1, a first member 10 has a fluid channel 11 therein and communicates with the outside through openings 12, 13, 14, and 15. An expanding member 16, which has a through-hole therein, is connected to the opening 13. A tube 17, which constitutes a supply channel, is fit into the through-hole to connect the fluid channel 11 in the first member 10 to an external device (not shown), such as a pump or a valve.

In the first embodiment, the tube 17 connected to a pump or a valve is the second member. The fluid to be supplied is not limited to liquid and may be gas or gel.

The tube 17 may be attached to the expanding member 16 or may be integrated with an expanding member. When the tube 17 is attached to the expanding member 16, the length of the tube 17 to be fit into the through-hole is not limited as long as the supply channel in the tube 17 communicates with the fluid channel 11.

The expanding member 16 according to the first embodiment is made of a material having a thermal expansion rate greater than that of the tube 17 and the first member 10.

The expanding member 16 connects the first member 10 and the second member, that is, the tube 17. The first member 10 and the second member may be secured to each other and immobilized or may be connected such that movement in a predetermined range is possible.

The first member 10 and the second member are firmly connected in the fluidic device according to the first embodiment as a result of expansion of the expanding member 16. Thus, when at least the expanding member 16 is expanded, fluid leakage from the connecting part of the first member 10 and the second member can be suppressed.

Since the expanding member 16 of the fluidic device according to the first embodiment expands, fluid leakage can be suppressed even when the size of a connecting portion of the first member 10 is different from that of the second member.

Since the expanding member 16 according to the first embodiment is made of a material that has a large expansion rate and expands/contracts in response to a temperature change, the volume of the expanding member 16 is small at a low temperature and is large at a high temperature.

The expanding member 16 that expands in response to a temperature change may be a member whose volume changes in response to the temperature change. For example, the expanding member 16 may be a member that has a large volume at a low temperature and a small volume at a high temperature.

The expansion rate of the expanding member 16 according to the first embodiment may larger than at least that of the first member 10. It is, however, preferable that the expansion rate of the expanding member 16 be larger than that of the second member.

For example, if the first member 10 is made of silica glass, the expanding member 16 may be made of a material having an expansion coefficient of 1.0×10⁻⁵ (/K) or greater or, more preferably, 1.0×10⁴ (/K) or greater.

The expanding member 16 is connected to the fluidic device under a temperature lower than the ambient temperature under which the fluidic device is to be used. In this way, while the fluidic device is being used, the volume of the expanding member 16 increases and the expanding member 16 contacts the fluidic device, suppressing fluid leakage.

The expanding member 16 of the fluidic device according to the first embodiment may be cooled by employing any cooling method and using a cooling apparatus or a temperature controlling apparatus.

As illustrated in FIG. 1, the expanding member 16 is prepared into a size that fits into the opening 13. Thus, even when the openings 12 and 14 are formed in the vicinity of the opening 13, the openings 12 and 14 are less likely affected by the expanding member 16.

The first member 10 and the expanding member 16 are tightly in contact as a result of a volume change caused by thermal expansion, and other connecting members are not used. Therefore, even when an opening is formed near another opening, the openings are less likely affected from each other, and thus, integration of the openings is possible.

The material of the first member 10 according to the first embodiment may be a glass material, such as silica glass or Pyrex glass, a polymer material, such as an acrylic material or polycarbonate, a semiconductor material, such as silicon, or a ceramic material, but is not limited as long as a fluid channel can be formed inside.

The first member 10 according to the first embodiment may be prepared using a known method. For example, to prepare the fluidic device with silica glass, acid etching may be employed.

In such a case, the first member 10 may be prepared by bonding together an upper surface substrate and a lower surface substrate. The upper surface and the lower surface correspond to the upper surface and the lower surface of the fluid channel.

For example, the lower-surface substrate may be made of silica glass and etched using an acid, such as hydrofluoric acid, to form a fluid channel. Then, the upper surface of the lower-surface substrate may be covered with another silica-glass substrate to form the first member 10.

The first member 10 of the fluidic device according to the first embodiment may be formed by bonding together two or three or more members.

The materials of the first member 10 and the second member of the fluidic device according to the first embodiment may be selected in accordance with the chemical resistance that the analytes have or the suitable way of detecting the analytes. It is, however, desirable to select materials that have a small thermal expansion rate.

The fluid channel 11 may be designed arbitrarily, and the design thereof is not limited to that illustrated in FIG. 1.

The size of the openings 12 to 15 is not limited but is determined in accordance with the size of the first member 10 and the type of fluid to be injected.

The cross-sectional area of the expanding member 16 may be the same as the area of the openings in the surface of the first member 10.

Since the first member 10 and the second member are firmly connected by the expansion of the expanding member 16, as described above, even when there is a difference in the areas, the connecting force of the first member 10 and the second member is not significantly affected.

The diameter of the fluid channel 11 in the fluidic device according to the first embodiment is not particularly limited. However, if the diameter is small, the fluidic device can be used as a microfluidic device. In such a case, it is preferable that the diameter be in the range of several micrometers to several hundred micrometers.

The height of the expanding member 16 according to the first embodiment can be set arbitrarily. It is preferable that the height be greater than the depth of the opening. This is to prevent the expanding member 16 from being buried in the opening and causing difficulty to be handled.

The material of the expanding member 16 according to the first embodiment is desirably an elastic material, such as an elastomer. Specifically, the material may be silicone-based rubber. If the expanding member 16 is made of an elastic material, it will tightly contact the inner wall of the opening when thermally expanded.

In particular, it is desirable that the elastic material be capable of contacting minute bumps on the inner wall of the opening because it is difficult to polish the inner wall. If a material that has poor elasticity is used, the expanding member 16 does not tightly contact the minute bumps. Specifically, the material may be a resin including polymers, such as polydimethylsiloxane.

The through-hole in the expanding member 16 according to the first embodiment may have a blocking prevention member that prevents the through-hole from being blocked. The blocking prevention member is constituted of a member desirably having a small thermal expansion rate.

The blocking prevention member is also referred to as a “spacer.” Here, “a small thermal expansion rate” refers to a thermal expansion rate smaller than that of the material of the expanding member 16.

For example, as a method of producing the expanding member 16 having a blocking prevention member, a hollow space may be formed in a polyetheretherketone (PEEK) resin member, and then the member may be coated with an elastomer.

The expanding member 16 or the coating agent are typically made of polymer-based material that has a large thermal expansion rate. More preferably, the expanding member 16 is made of a material having a thermal expansion rate greatly different from the thermal expansion rate of the material of the fluidic device.

The tube 17 according to the first embodiment is, for example, made of polytetrafluoroethylene or a polyether-ketone-based material but is not limited thereto. Instead of a tube, a glass capillary or a metal needle may be used.

Fluid is supplied from the outside of the fluidic device through the tube 17, which is the second member.

The material and size of the fluidic device and the expanding member 16 can be selected in accordance with the ambient temperature at which the fluidic device is to be used.

The expanding member 16 in the fluidic device according to the first embodiment utilizes the property of thermal expansion of substances.

Most substances, except for some certain substances and substances in certain temperature ranges, experience a volume increase when the ambient temperature of the substance increases.

The rate of expansion is represented by a thermal expansion coefficient, and each substance has its corresponding coefficient. For example, the thermal expansion coefficient of aluminum is approximately 2.5×10⁻⁵ (/K), and thermal expansion coefficient of silica glass is approximately 5.6×10⁻⁷ (/K).

The thermal expansion coefficient includes a linear expansion coefficient (α) and an volumetric expansion coefficient (β), which satisfy the relationship β=3α. In the first embodiment, the thermal expansion coefficient indicates a linear expansion coefficient, unless otherwise noted.

In the fluidic device according to the first embodiment, the first member 10 and the second member are connected with the expanding member 16 by thermal expansion alone. Thus, an adhesive is not used.

Since the first embodiment does not use a process of forming a hallow protrusion that communicates with an opening in the fluidic device or a process of applying an adhesive material, the production process is simplified.

If an adhesive is used, the adhesive may enter the fluid channel 11, but the expanding member 16 according to the first embodiment does not enter the fluid channel and does not use an adhesive. Thus, the fluid channel 11 is not blocked by an adhesive and fluidic devices are not wasted.

The production process is simplified because the fluidic device does not require the processes of producing a protrusion and applying an adhesive substance.

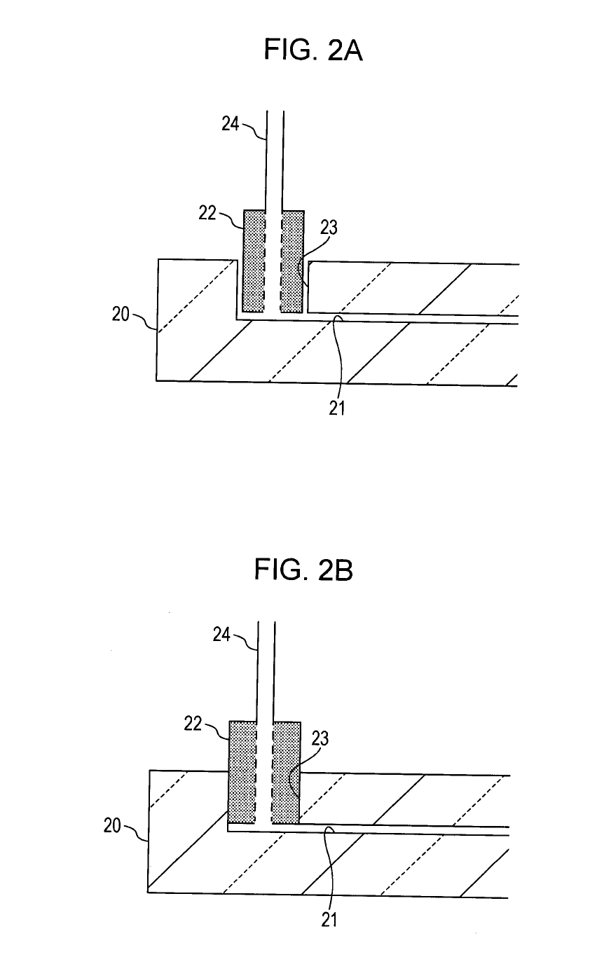

Application of an expanding member according to the first embodiment will be described below with reference to FIGS. 2A and 2B.

FIGS. 2A and 2B are schematic sectional view illustrating an opening, into which an expanding member is fit, toward a fluid channel. The opening corresponds to the opening 13 in FIG. 1, into which the expanding member 16 is fit, and the fluid channel corresponds to the fluid channel 11 in FIG. 1.

In FIGS. 2A and 2B, a first member 20 has a fluid channel 21 therein. The first member 20 has an opening 23 in the surface, and an expanding member 22 is fit into the opening 23. A tube 24 is connected to the first member 20 with the expanding member 22.

The expanding member 22 has a through-hole through which the tube 24 and the first member 20 communicate.

The circumference of the expanding member 22 is substantially the same shape and size as the outline of the cross-section of the opening 23 in the surface of the first member 20.

The first member 20 according to the first embodiment is made of silica glass, and the expanding member 22 is made of polydimethylsiloxane (PDMS). Silica glass has a small thermal expansion coefficient, that is, approximately 5.6×10⁻⁷ (/K), whereas PDMS has a large thermal expansion coefficient, that is, approximately 3.1×10⁻⁴ (/K).

PDMS is a substance that is normally a liquid but can be processed into a desired shape by adding a thermal curing agent.

The amount of thermal curing agent to be added is determined based on the shape of the expanding member 22 and is preferably an amount that does not interfere with the expansion of the expanding member 22.

The expanding member 22 according to the first embodiment has a cross-section that is the same as the cross-section of the opening 23. The height of the expanding member 22 is greater than the depth of the opening 23.

The expanding member 22 according to the first embodiment is produced by preparing a mold of the expanding member 22, injecting PDMS and a thermal curing agent in the mold, and curing by heating the mold for about 1 to 48 hours at a temperature in the range of room temperature to approximately 80° C.

FIG. 2A illustrates the first member 20 in an environment at a temperature lower than the temperature of the environment in which analysis and chemical synthesis are to be carried out.

Under this condition, the expanding member 22 fit into the opening 23 shrinks due to low temperature, preventing the opening 23 from being sufficiently sealed.

For example, when the temperature of PDMS is decreased by 10° C., its volume shrinks to approximately 99% of the original volume. As a result, a gap forms between the inner wall of the opening 23 and the expanding member 22, allowing the expanding member 22 to be fit into the opening 23.

When the temperature is increased again so as to use the fluidic device, the expanding member 22 expands and tightly contacts the inner wall of the opening 23, as illustrated in FIG. 2B, preventing the fluid from leaking.

Due to thermal expansion, the expanding member 22 also expands inward in the through-hole, and as a result, the tube 24 is even more firmly held.

In this way, external fluid supplied to the first member 20 does not leak at the opening 23 and flows into the fluid channel 21.

The tube 24 can be easily removed, when necessary, by lowering the ambient temperature again.

In the first embodiment, the opening 23 is formed in the upper surface of the first member 20, and the tube 24 is inserted from above. That is, the path from the opening 23 to the fluid channel 21 is bent in the first member 20.

When the path from the opening 23 to the fluid channel 21 is bent, the direction of the fluid flowing through the fluid channel 21 in the first member 20 intersects with the direction of the opening 23 formed in the thickness direction of the first member 20.

When the path from the opening 23 to the fluid channel 21 is bent, the fluidic device according to the first embodiment will have a fluid channel extending in the direction of the path after reaching the bent section and an opening that is disposed in the direction of the path before reaching the bent section.

When the thickness direction of the flat first member 20 is defined as an up-and-down direction, the opening 23 opens upward, and the expanding member 22 contacts the inner wall of the opening 23.

When the expanding member 22 expands and connects the first member 20 and the second member, it is desirable that the path in the first member 20 from the opening 23 to the fluid channel 21 not be blocked. Thus, the expanding member 22 may be expanded while being held in such a manner that it does not contact the bottom surface of the opening 23.

When the path from the opening 23 to the fluid channel 21 is bent in the first member 20 according to the first embodiment, it is desirable that the opening 23 in the first member 20 have an inner-wall part that prevents the first member 20 from blocking the path from the opening 23 to the fluid channel 21.

Such an inner-wall part may be provided by forming a step on the inner wall of the opening 23, tapering the inner wall, or providing a protrusion on the inner wall.

The fluidic device according to the first embodiment may have the opening 23 in the side surface of the first member 20, and the supply channel in the second member may be fit into the first member 20 from the side. That is, the position of the opening 23 in the first member 20 is not limited.

### Second Embodiment

In a second embodiment, the second member is a substrate having a supply channel.

The second embodiment is the same as the first embodiment, except that the second member is a substrate having a supply channel. The supply channel may be the same as the fluid channel in the first member.

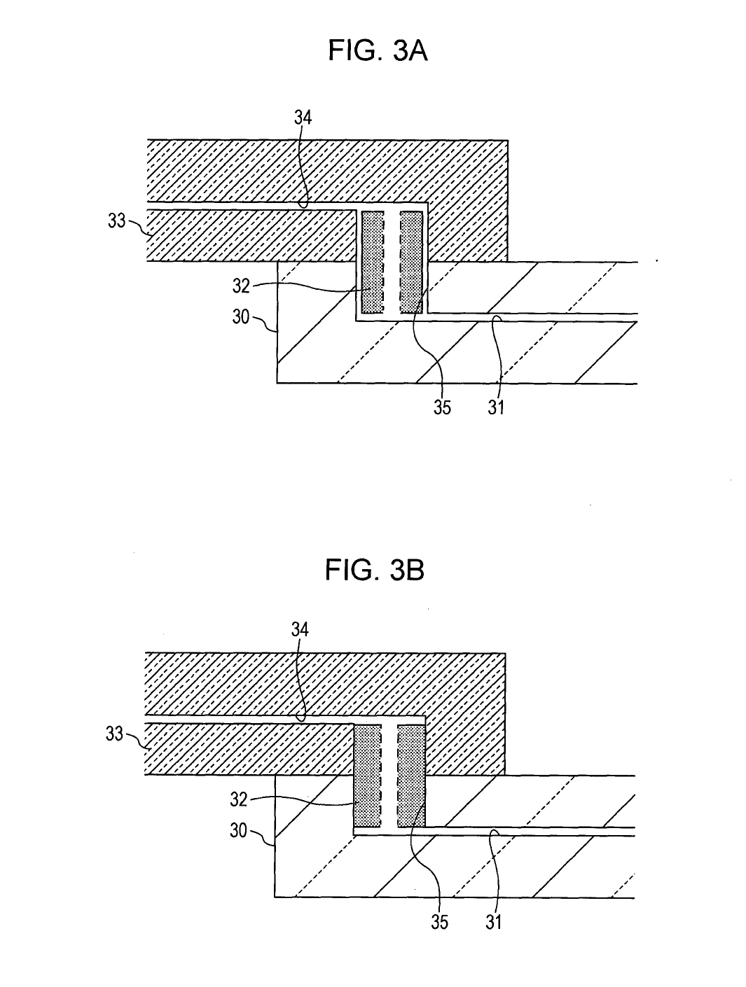

FIG. 3A illustrates a first member 30 and a second member 33 placed in an environment of a temperature lower than that of the environment in which analysis and chemical synthesis are to be carried out. Under this condition, an expanding member 32, which is fit into an opening 35, shrinks due to low temperature, preventing the opening 35 from being sufficiently sealed.

The temperature is then increased again for use of the first member 30 and the second member 33. As a result, as illustrated in FIG. 3B, the expanding member 32 expands and tightly contacts the inner wall of the opening 35, forming a seal against the fluid leakage.

Due to thermal expansion, the expanding member 32 also expands inward in the through-hole, but a blocking prevention member (not shown) prevents the through-hole from being blocked.

In this way, a fluid channel 31 in the first member 30 communicates with a fluid channel 34 in the second member 33, and a fluid can flow therein without leaking.

The expanding member 32 according to the second embodiment may be included in the first member 30 or the second member 33.

It is desirable that the opening in the second member 33 of the fluidic device according to the second embodiment has an inner-wall part that prevents the path from the opening to the fluid channel 34 from being blocked by the expanding member 32.

The fluidic device according to the second embodiment is not limited to such a configuration, and, instead, the opening may be formed in a side surface of the first member 30 or a side surface of the second member 33.

### Third Embodiment

In a third embodiment, a first member has a plurality of openings.

The third embodiment is the same as the first embodiment, except that the first member has a plurality of openings and a plurality of second members are provided, or that the second member has a plurality of openings.

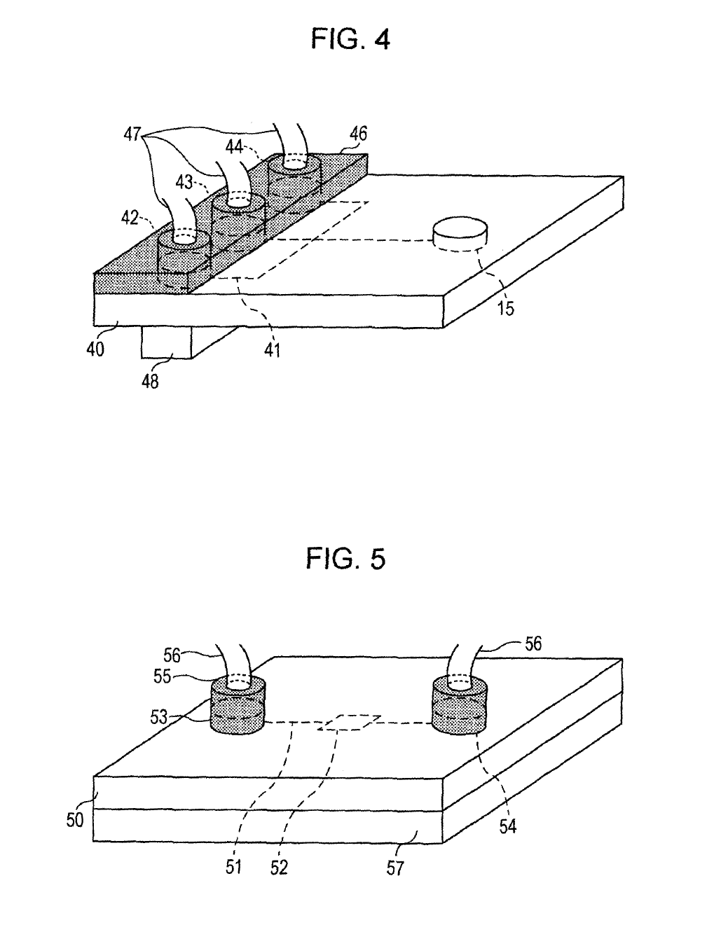

As illustrated in FIG. 4, a temperature-lowering device 48 is disposed on the bottom surface of a first member 40 to lower the temperature near openings 42, 43, and 44 when an expanding member 46 is fit into the openings 42, 43, and 44. The temperature-lowering device may also be referred to as a cooling device.

A fluidic device according to the third embodiment has a plurality of openings and a plurality of fluid channels. The fluid channels may be independent from each other or may merge into a single fluid channel inside the first member 40.

When the second member according to the third embodiment is a substrate that has a plurality of supply channels, the fluid channels in the first member 40 and the supply channels in the second member are connected.

It is also possible to connect the plurality of supply channels to the single fluid channel in the first member 40.

The fluid channel in the fluidic device according to the third embodiment may have a chemical-reaction region where the fluid yields a chemical reaction.

The fluidic device may constitute a chemical reaction system including an image capturing device configured to capture an image of the chemical-reaction region.

When the fluidic device is used as a chemical reaction system, the plurality of fluid channels in the first member 40 are integrated into a single fluid channel so as to cause a chemical reaction.

When the fluidic device according to the third embodiment has a plurality of openings, the positions of the openings are not limited. For example, the openings may be aligned parallel to an edge of the first member 40 or may be arranged in a zigzag pattern.

It is desirable that the fluidic device according to the third embodiment have a plurality of openings because the first member 40 and the second member will less likely be misaligned in the rotating direction.

When the first member 40 and the second member are connected by a plurality of expanding members 46, the connection becomes even tighter.

### Fourth Embodiment

In the fourth embodiment, a method of inserting an expanding member when the fluidic device is used at a temperature higher than room temperature (approximately 25° C.) will be described.

The fourth embodiment is the same as the first embodiment, except that the first member or the second member has a temperature controlling apparatus 57.

FIG. 5 illustrates the temperature controlling apparatus 57, which is disposed under a first member 50. The temperature controlling apparatus 57 maintains a constant temperature and is, for example, a hot plate or a Peltier device.

The temperature controlling apparatus 57 does not necessarily have to be in contact with the first member 50. Instead, the fluidic device may be disposed inside an oven or an incubator that can maintain a constant temperature and function as a temperature controlling apparatus.

The fluidic device is typically used under a condition a temperature of which is higher than room temperature in the processes of gene amplification or nucleic acid amplification, such as cell culture, isothermal and chimeric primer-initiated amplification of nucleic acids (ICAN), and polymerase chain reaction (PCR), but the use is not limited to such purposes.

Expanding members 55 each having a cross-section that is substantially the same size and shape as openings 53 and 54 is fit into the openings 53 and 54 in advance. Thus, the ambient temperature does not have to be lowered, and the expanding members 55 are inserted at room temperature.

Then, the first member 50 is set under a predetermined temperature condition using the temperature controlling apparatus 57. As a result, the expanding members 55 expand and tightly contact the inner walls of the corresponding openings 53 and 54, forming seals between tubes 56 and the inner walls of the openings 53 and 54.

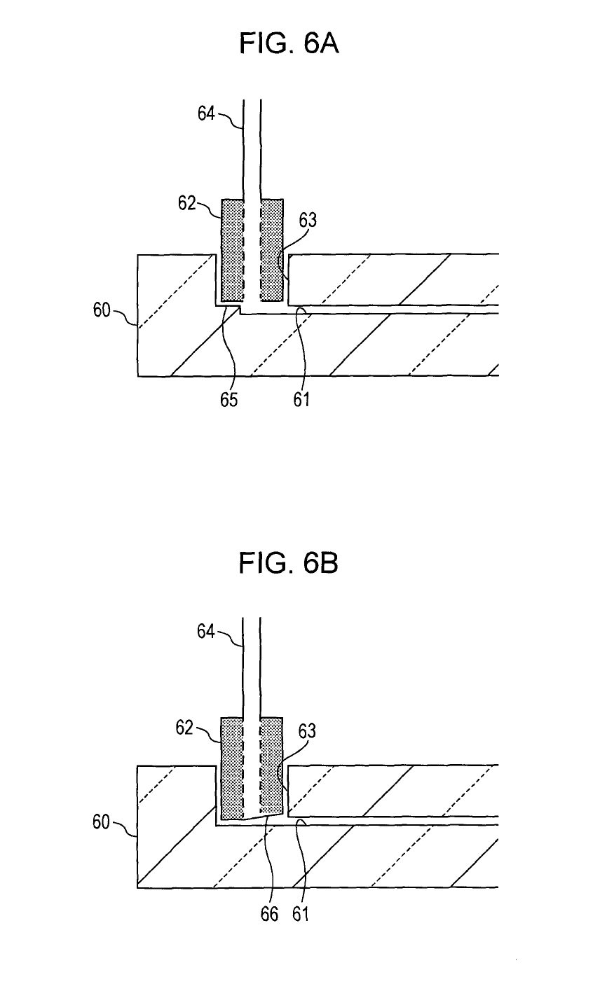

FIG. 6A illustrates a step-like part 65, which is an example of an inner-wall part that prevents a fluid channel from being blocked by an expanding member of the fluidic device according to an embodiment.

FIG. 6B illustrates a notch 66 formed in the bottom surface of an expanding member for preventing a fluid channel from being blocked. The shapes and structures illustrated in FIGS. 6A and 6B are merely examples as a result of processing conducted to prevent the entire bottom surface of the expanding member from contacting the bottom surface of the opening and are not limited thereto.

The fluidic device according to an embodiment can be used in μ-TAS.

In particular, the fluidic device can be suitably used in a measurement apparatus that requires a temperature higher than room temperature during measurement or reaction. Polymerase chain reaction (PCR) is an example of such a reaction.

A fluidic device used for a PCR process may have, at different positions, an amplification region in which the target DNA is amplified and a measurement region in which the temperature of DNA melting is measured.

That is, the fluidic device according to an embodiment may be a nucleic-acid analyzing system having an amplification region for amplifying nucleic acid and a measurement region for measuring the melting temperature of nucleic acid.

The nucleic-acid analyzing system according to an embodiment may include an image capturing device for capturing images of the amplification region and the measurement region. A plurality of image capturing devices may be provided and may be integrated on a single chip.

In an image sensor including a plurality of image capturing devices, one image capturing device may be used to capture images of both the amplification region and the measurement region.

A reaction in the fluid channel can be captured by forming the fluidic device with a material having a high transmittance for visible light. For example, the fluidic device may be used as an analyzer that includes an image capturing apparatus for capturing an image of a reaction with light.

When the fluidic device according to an embodiment is used as a measurement apparatus for measuring reaction with light, it is desirable that the first member be made of a light-transmissive material.

An image of the inside of the fluid channel can be captured from the upper or lower surface by an image capturing apparatus including an image capturing element.

It is more desirable that the first member be prepared by combining a light-transmissive member and a light-reflective member so as to efficiently capture an image of the inside of the fluid channel.

In such a case, the upper or lower surface of the first member is made of a light-transmissive member, and the other surface is made of a light-reflective member.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-105634, filed May 10, 2011, which is hereby incorporated by reference herein in its entirety.

### REFERENCE SIGNS LIST

10 first member
11 fluid channel
12, 13, 14, 15 opening
16 expanding member
17 tube (second member)
48 temperature-lowering device
50 first member
51 fluid channel
52 chamber
56 tube
57 temperature controlling apparatus

1. A fluidic device comprising:
a first member having a fluid channel communicating with an opening; and
a second member having a supply channel supplying a fluid to the fluid channel through the opening, wherein
an expanding member having a through-hole is fit into the opening,
the fluid channel and the supply channel communicate with each other through the through-hole in the expanding member, and
the first member and the second member are connected to each other with at least the expanding member being in an expanded state.

2. The fluidic device according to claim 1, wherein the expanding member has a blocking prevention member provided on an inner side of the through-hole and configured to prevent blocking of the through-hole due to expansion of the expanding member.

3. The fluidic device according to claim 1, wherein
a path from the opening to the fluid channel is bent, and
the opening has an inner-wall part shaped to prevent the expanding member from blocking the path.

4. The fluidic device according to claim 3, wherein the inner-wall part has a step or a tapered shape.

5. The fluidic device according to claim 1, wherein
the first member has at least a plurality of openings,
the second member has at least a plurality of supply channels, and
the plurality of openings and the plurality of supply channels are connected and communicate with each other.

6. The fluidic device according to claim 1, wherein the fluid channel has a chemical-reaction region where a fluid yields a reaction.

7. A chemical reaction system that yields a chemical reaction using the fluidic device according to claim 6, the system comprising:
an image capturing device configured to capture an image of the chemical-reaction region.

8. A nucleic-acid analyzing system that analyzes nucleic acid using the fluidic device according to claim 1, wherein
the fluidic device has an amplification region where nucleic acid is amplified and a measurement region where a nucleic acid melting temperature is measured, and
the nucleic-acid analyzing system includes an image capturing device configured to capture images of the amplification region and the measurement region.

9. The nucleic-acid analyzing system according to claim 8, wherein the first member is a light transmissive member.

10. The nucleic-acid analyzing system according to claim 8, wherein the nucleic-acid analyzing system includes a plurality of image capturing devices, and at least one of the image capturing devices captures the amplification region and at least another image capturing device captures the measurement region.

11. The nucleic-acid analyzing system according to claim 10, wherein the image capturing device capturing the amplification region and the image capturing device capturing the measurement region are integrated on a single chip.
